(19) United States
(12) Patent Application Publication
(10) Pub. No.: US 2011/0091899 A1
(43) Pub. Date: Apr. 21, 2011

(54) COMBINATION OF RISK ALLELES ASSOCIATED WITH AUTISM

(75) Inventors: Jörg Hager, Mennecy (FR); Frédéric Tores, Lieusaint (FR); Francis Rousseau, Savigny sur Orge (FR)

(73) Assignee: Integragen, Evry (FR)

(21) Appl. No.: 12/997,150

(22) PCT Filed: Jun. 12, 2009
(86) PCT No.: PCT/EP2009/057271

§ 371 Date: Dec. 9, 2010

Related U.S. Application Data
(60) Provisional application No. 61/060,945, filed on Jun. 12, 2008.

(51) Int. Cl. C12Q 1/68

(52) U.S. Cl. 435/6

(57) ABSTRACT

The present invention relates to a method of detecting the presence of or predisposition to autism, or to an autism spectrum disorder, the method comprising detecting the presence of an alteration in the gene loci PITX1, ATP2B2, SLC25A12 and EN2 in a sample from said subject. More particularly, the presence of specific single nucleotide polymorphisms (SNPs) within these genes correlates to a substantially increased risk to develop autism.

The present invention relates to a method for detecting the presence or predisposition to autism, by detecting a combination of risk alleles in several genes simultaneously.

### BACKGROUND OF THE INVENTION

Autism is a developmental disorder characterized by impairments in social interaction and communication associated with repetitive patterns of interest or behavior (Filipek et al. 1999). Autism marks a severe clinical diagnosis within a spectrum of pervasive developmental disorders including Rett syndrome, Asperger syndrome and other non-specified developmental disorders.

Depending on the clinical criteria and the geographical location estimations of the prevalence of autism vary between 0.05 to 0.6% (Chakrabarti et al. 2001; Fombonne 2003). Autism shows a well established gender distortion with about four times as many males than females being affected (Fombonne et al. 2003). Monozygotic and dizygotic twin studies have shown that autism has a significant genetic component with monozygotic twin concordance rates as high as 91% if broad diagnostic criteria are applied. Autism does not follow a simple Mendelian inheritance pattern and this is thought to be due to the involvement of multiple genes (Veenstra-VanderWeele et al. 2004).

The diagnosis of autism is not unified and a number of distinct criteria are applied in different parts of the world. In many European countries diagnostic criteria like DSM-IV for psychiatric diseases are applied. The ADI-R and ADOS tests, mainly applied in the US, have become a kind of gold standard and are more and more implemented in Europe as well.

The ADI-R is a standardized, semi-structured clinical review for caregivers of children and adults (Lord et al. 1994). The interview contains 111 items and focuses on behaviors in three content areas: quality of social interaction, (e.g., emotional sharing, offering and seeking comfort, social smiling and responding to other children); communication and language (e.g., stereotyped utterances, pronoun reversal, social usage of language); and repetitive, restricted and stereotyped interests and behavior (e.g., unusual preoccupations, hand and finger mannerisms, unusual sensory interests). The measure also includes other items relevant for treatment planning, such as self-injury and over activity. Responses are, scored by the clinician based on the caregiver's description of the child's behavior. Questions are organized around content area, and definitions of all behavioral items are provided. Within the area of Communication, for example, “Delay or total lack of language not compensated by gesture” is further broken down into specific behavioral items: pointing to express interest, conventional gestures, nodding head, and head shaking. Similarly, within the area of Reciprocal Social Interaction, in lack of socio-emotional reciprocity and modulation to context includes the following behaviors: use of other's body, offers comfort, inappropriate facial expressions, quality of social overtures, and appropriateness of social response.

This interviewer-based instrument requires substantial training in administration and scoring. A highly trained clinician can administer the ADI-R to the parent of a 3- or 4-year old suspected of autism in approximately 90 minutes. The interview may take somewhat longer when administered to parents of older children or adults.

In a study of 51 autistic and 43 non-autistic mentally handicapped preschoolers, using similar procedures to the study described above, weighted kappas for inter-rater reliability (calculated using percent exact agreement) ranged from 0.62 to 0.96. Test-retest reliabilities, using intra-class correlations, were above 0.90 in all domains and sub-domains. A reliability study of the German form of the ADI-R with 22 individuals ages 5 to 29 (mean age=13.5 years) demonstrated high levels of inter-rater reliabilities (intra-class correlations) for all three domains: Reciprocal social interaction=0.75; Communication and language=0.77; and Repetitive and stereotyped behaviors and interests=0.80.

The ADI-R is a semi-structured instrument for diagnosing autism in children and adults with mental ages of 18 months and above. The instrument has been shown to be reliable and to successfully differentiate young children with autism from those with mental retardation and language impairments. The revised version of the instrument has been tested primarily with parents of preschoolers presenting for the first time with possible autism. In this population, the algorithms based on DSM-IV and ICD-10 criteria have been shown to have high levels of sensitivity and moderate levels of specificity.

The greatest difficulty is in the over diagnosis of autism in young, severely mentally handicapped children. In one study, nearly 60% of the non-autistic children with no speech at all met criteria for autism in each of the three diagnostic areas. All of these children had mental ages below 18 months. Items concerning communication do not appear to be useful in differentiating autistic preschoolers from other severely language-delayed children. Further research is required to test the ability of the ADI-R to discriminate between children with autism and other pervasive developmental disorders. The utility of the instrument for monitoring treatment effects is unknown.

There is no drug therapy available for autism, although some autistic individuals have been treated with anti-depressant drugs (eg Prozac) for secondary symptoms. The main treatments proposed are based on intensive educational programs. Applied early enough some studies show that as many as 50% of autistic children participating in those programs can be referred back to normal schooling and education. In a recent UK study the potential socio-economic benefit of early intensive treatment has been estimated to be as high as 1.8 million £ per patient over the life time of the patient. The age at which the therapy is proposed is of significant importance. Ideally the programs should start at 18 months age. As outlined above the ADI-R cannot be used for diagnosis under the age of 18 months. Indeed, for infrastructural (availability of trained experts, in the US only 10% of suspected autistic children have direct access to specialists able to carry out ADI-R) and social reasons the average age of diagnosis is 5 years in the US and 8 years in France. A genetic test would have a huge impact, because the test can easily be applied at any age (e.g. after birth) and can be used for prescreening of individuals for eligibility for an ADI-R, thereby substantially shortening the time from diagnosis to treatment.

### SUMMARY OF THE INVENTION

Autism is highly influenced by genetic factors. Several genes associated with autism have been identified by academic groups and through in-house research efforts at IntegraGen SA (IntegraGen). However, the contribution to disease risk of each individual gene identified is generally low, and the odds ratio per risk allele rarely is above 1.5. Thus, the predictive power for each gene individually is too small to be of clinical utility in complex diseases. The invention described here led to the identification and choice of a combination of four (4) genes, Paired-like homeodomain transcription factor 1 (PITX1), Plasma membrane calcium ATPase 2 (ATP2B2), Solute carrier family 25 (mitochondrial carrier, Aralar) member 12 (SLC25A12), and Engrailed 2 (EN2), to analyze in a multigene autism risk assessment model. In particular, genotyping these four genes can allow the estimation of a predictive value for the risk of developing autism in yet non-affected siblings of affected individuals. The four genes were chosen from a larger panel of genes as to maximize the predictive value of a genetic test. The inventors showed that the predictive value that is obtained by detecting combinations of polymorphisms in these genes is superior to the predictive value obtained when observing alterations in each gene separately, demonstrating its clinical validity.

The clinical utility of this test resides in its ability to select at risk individuals for earlier down-stream diagnosis using psychological profiling tests (e.g. ADI-R or ADOS). The test may also be used in affected individuals to accompany these profiling tests to substantiate the diagnosis for autism and distinguish it from other psychiatric conditions.

### LEGENDS TO THE FIGURES

FIG. 1 shows an increase in risk associated with increasing numbers of risk alleles. Four single nucleotide polymorphisms (rs6872664 [PITX1], rs35678 [ATP2B2], rs2292813 [SLC25A12], and rs1861972 [EN2]) were analyzed in a multigene risk assessment model using all siblings to the proband and a broad definition of autism, which includes autism spectrum disorders and pervasive developmental disorders. Odds ratios and 90% confidence intervals (CI) are presented. Data are adjusted for gender. The one-sided p-value was p=0.002.

FIG. 2 is a Receiver operator characteristic curve for risk alleles of rs6872664 (PITX1), rs35678 (ATP2B2), rs2292813 (SLC25A12), and rs1861972 (EN2). The area under the curve was 0.59 (95% CI: 0.54-0.63, p=0.001).

### DETAILED DESCRIPTION OF THE INVENTION

The invention provides a method of detecting the presence of or predisposition to autism, or to an autism spectrum disorder in a subject, the method comprising detecting the presence of an alteration in the gene loci of at least PITX1, ATP2B2, SLC25A12 and EN2 in a sample from said subject.

In a preferred embodiment, the alteration is a single nucleotide polymorphism.

Autism is typically characterized as part of a spectrum of disorders (ASDs) including Asperger syndrome (AS) and other pervasive developmental disorders (PPD). Autism is construed as any condition of impaired social interaction and communication with restricted repetitive and stereotyped patterns of behavior, interests and activities present before the age of 3, to the extent that health may be impaired. AS is distinguished from autistic disorder by the lack of a clinically significant delay in language development in the presence of the impaired social interaction and restricted repetitive behaviors, interests, and activities that characterize the autism-spectrum disorders (ASDs). PPD-NOS (PPD, not otherwise specified) is used to categorize children who do not meet the strict criteria for autism but who come close, either by manifesting atypical autism or by nearly meeting the diagnostic criteria in two or three of the key areas.

The invention provides diagnostic screening methods based on a monitoring of several genes in a subject. The subject may be at early, pre-symptomatic stage, or late stage. The subject may be any human male or female, preferably a child or a young adult. The subject can be asymptomatic.

The method is particularly useful when the subject is a sibling of an individual with autism or an autism-spectrum disorder, i.e. an individual already diagnosed with autism or an autism spectrum disorder. The likelihood that a sibling of a child with autism also develops autism is between 3 and 6 percent (Chakrabarti & Fombonne, 2001). This is approximately 20 times greater than the rate at which autism affects individuals who are not related to an affected individual. The method of the invention can be performed at any age after birth and used to pre-screen individuals requiring further assessment with the ADI-R, shortening the time from diagnosis to intervention.

The diagnosis methods can be performed in vitro, ex vivo or in vivo, preferably in vitro or ex vivo. They use a sample from the subject. The sample may be any biological sample derived from a subject, which contains nucleic acids. Examples of such samples include fluids, tissues, cell samples, organs, biopsies, etc. Most preferred samples are blood, plasma, saliva, urine, seminal fluid, etc. The sample may be collected according to conventional techniques and used directly for diagnosis or stored. The sample may be treated prior to performing the method, in order to render or improve availability of nucleic acids or polypeptides for testing.

Treatments include, for instant, lysis (e.g., mechanical, physical, chemical, etc.), centrifugation, etc. Also, the nucleic acids may be pre-purified or enriched by conventional techniques, and/or reduced in complexity. Nucleic acids may also be treated with enzymes or other chemical or physical treatments to produce fragments thereof. Considering the high sensitivity of the claimed methods, very few amounts of sample are sufficient to perform the assay.

The sample is preferably contacted with reagents such as probes, or primers in order to assess the presence of an altered gene locus. Contacting may be performed in any suitable device, such as a plate, tube, well, glass, etc. In specific embodiments, the contacting is performed on a substrate coated with the reagent, such as a nucleic acid array. The substrate may be a solid or semi-solid substrate such as any support comprising glass, plastic, nylon, paper, metal, polymers and the like. The substrate may be of various forms and sizes, such as a slide, a membrane, a bead, a column, a gel, etc. The contacting may be made under any condition suitable for a complex to be formed between the reagent and the nucleic acids of the sample. The finding of a specific allele of PITX1, ATP2B2, SLC25A12 and EN2 DNA in the sample is indicative of the presence of a gene locus variant in the subject, which can be correlated to the presence, predisposition or stage of progression of autism, or an autism spectrum disorder. For example, an individual having a germ line mutation has an increased risk of developing autism, an autism spectrum disorder, or an autism-associated disorder. The determination of the presence of an altered gene locus in a subject also allows the design of appropriate therapeutic intervention, which is more effective and customized. Also, this determination at the pre-symptomatic level allows a preventive regimen to be applied.

An alteration in a gene locus may be any form of mutation(s), deletion(s), rearrangement(s) and/or insertions in the coding and/or non-coding region of the locus, alone or in various combination(s). Alterations more specifically include point mutations or single nucleotide polymorphisms (SNP). Deletions may encompass any region of two or more residues in a coding or non-coding portion of the gene locus, such as from two residues up to the entire gene or locus. Typical deletions affect smaller regions, such as domains (introns) or repeated sequences or fragments of less than about 50 consecutive base pairs, although larger deletions may occur as well. Insertions may encompass the addition of one or several residues in a coding or non-coding portion of the gene locus. Insertions may typically comprise an addition of between 1 and 50 base pairs in the gene locus. Rearrangement includes inversion of sequences. The gene locus alteration may result in the creation of stop codons, frameshift mutations, amino acid substitutions, particular RNA splicing or processing, product instability, truncated polypeptide production, etc. The alteration may result in the production of a polypeptide with altered function, stability, targeting or structure. The alteration may also cause a reduction in protein expression or, alternatively, an increase in said production.

Once a first SNP has been identified in a genomic region of interest, more particularly in ATP2B2 gene locus, the practitioner of ordinary skill in the art can easily identify additional SNPs in linkage disequilibrium with this first SNP. Indeed, any SNP in linkage disequilibrium with a first SNP associated with autism or an associated disorder will be associated with this trait. Therefore, once the association has been demonstrated between a given SNP and autism or an associated disorder, the discovery of additional SNPs associated with this trait can be of great interest in order to increase the density of SNPs in this particular region.

Identification of additional SNPs in linkage disequilibrium with a given SNP involves: (a) amplifying a fragment from the genomic region comprising or surrounding a first SNP from a plurality of individuals; (b) identifying of second SNPs in the genomic region harboring or surrounding said first SNP; (c) conducting a linkage disequilibrium analysis between said first SNP and second SNPs; and (d) selecting said second SNPs as being in linkage disequilibrium with said first marker. Subcombinations comprising steps (b) and (c) are also contemplated. Methods to identify SNPs and to conduct linkage disequilibrium analysis can be carried out by the skilled person without undue experimentation by using well-known methods.

These SNPs in linkage disequilibrium can also be used in the methods according to the present invention, and more particularly in the diagnostic methods according to the present invention.

### PITX1, ATP2B2, SLC25A12 and EN2 Genes

International patent application WO2006/003520 discloses that the PITX1 gene on chromosome 5 and certain alleles thereof are related to susceptibility to autism. As used herein, the term “PITX1 gene” designates the pituitary homeobox transcription factor 1 gene on human chromosome 5q31.1, as well as variants, analogs and fragments thereof, including alleles thereof (e.g., germline mutations) which are related to susceptibility to autism and autism-associated disorders. The PITX1 gene may also be referred to as paired-like homeodomain transcription factor pituitary homeobox 1, or PTX1.

International patent application WO2006/100608 describes that the ATP2B2 gene on chromosome 3 and certain alleles thereof are related to susceptibility to autism. As used herein, the term “ATP2B2 gene” designates the ATPase, Ca++ transporting, plasma membrane 2 gene on human chromosome 3p25.3, as well as variants, analogs and fragments thereof, including alleles thereof (e.g., germline mutations) which are related to susceptibility to autism and autism-associated disorders. The ATP2B2 gene may also be referred to as PMCA2.

International patent application WO2005/055807 discloses that the SLC25A12 gene on chromosome 2q24 and certain alleles thereof are related to susceptibility to autism. This gene is name after “Solute carrier family 25 member 12” and encodes a protein also known as Calcium-binding mitochondrial carrier protein (Aralar1) or calcium-dependent mitochondrial aspartate/glutamate carrier (AGC1).

International patent application WO2005/007812 discloses that the EN2 gene on chromosome 7q36.3 and certain alleles thereof are related to susceptibility to autism. This gene is name after “ENGRAILED 2”, a homeobox transcription factor.

In previous studies, rs6872664 (PITX1), rs35678 (ATP2B2), rs2292813 (SLC25A12), and rs1861972 (EN2) showed significant association with autism with relative risks varying with the gene, the definition of autism, and the genotype (heterozygous or homozygous).(Philippi et al, 2007; WO2006/100608, Ramoz et al, 2004; Benayed et al, 2005)

In a preferred embodiment, the method of the invention comprises detecting the presence of a single nucleotide polymorphism (SNP) at any of positions rs6872664, rs6596188, rs6596189 or rs6871427 of PITX1, and/or the presence of a single nucleotide polymorphism (SNP) at any of positions rs35678, rs3774180, rs775018, rs28113, rs2278556, or rs3774169 of ATP2B2, and/or the presence of a single nucleotide polymorphism (SNP) at any of positions rs2292813, rs13016580, rs3770459, or rs1996424 of SLC25A12 and/or detecting the presence of a single nucleotide polymorphism (SNP) at any of positions rs1861972, or rs1861973 of EN2 .

### PITX1

| | Position (Build | Position (Build | | | |
|---|---|---|---|---|---|
| rs ID | 35) | 35) | Allele 1 | Allele 2 | SEQ ID |
| rs6872664 | 134395487 | 134395507 | C = 1* | T = 2 | 1 |
| rs6596188 | 134395992 | 134396012 | A = 1* | T = 2 | 2 |
| rs6596189 | 134396058 | 134396078 | C = 1* | T = 2 | 3 |
| rs6871427 | 134401916 | 134401936 | C = 1 | G = 2* | 4 |

### ATP2B2

| | Position (Build | Position (Build | | | |
|---|---|---|---|---|---|
| rs ID | 35) | 35) | Allele 1 | Allele 2 | SEQ ID |
| rs35678 | 10354913 | 10354933 | C = 1 | T = 2* | 5 |
| rs3774180 | 10371978 | 10371998 | A = 1 | G = 2* | 6 |
| rs775018 | 10375135 | 10375155 | A = 1 | G = 2* | 7 |
| rs28113 | 10375633 | 10375653 | A = 1* | G = 2 | 8 |
| rs2278556 | 10377093 | 10377113 | A = 1* | G = 2 | 9 |
| rs3774169 | 10391277 | 10391297 | C = 1 | T = 2* | 10 |

### SLC25A12

| | Position (Build | Position (Build | | | |
|---|---|---|---|---|---|
| rs ID | 35) | 35) | Allele 1 | Allele 2 | SEQ ID |
| rs2292813 | 172469726 | 172469746 | C = 1* | T = 2 | 11 |
| rs13016580 | 172472025 | 172472045 | A = 1 | G = 2* | 12 |
| rs3770459 | 172474089 | 172474109 | G = 1 | T = 2* | 13 |
| rs1996424 | 172503862 | 172503882 | A = 1 | T = 2* | 14 |

### EN2

| | Position (Build | Position (Build | | | |
|---|---|---|---|---|---|
| rs ID | 35) | 35) | Allele 1 | Allele 2 | SEQ ID |
| rs1861972 | 154753459 | 154753479 | A = 1* | G = 2 | 15 |
| rs1861973 | 154753611 | 154753631 | C = 1* | T = 2 | 16 |

In a still preferred embodiment, the method comprises detecting the simultaneous presence of a SNP at position rs6872664 of P1TXL rs35678 of ATP2B2, rs2292813 of SLC25A12 and rs1861972 of EN2 .

The alleles are as follows: rs6872664 (major allele [risk allele]=C; minor allele=T); rs35678 (major allele=C; minor allele [risk allele]=T; recessive coding for risk allele in risk score), rs2292813 (major allele [risk allele]=C; minor allele=T), and rs1861972 (major allele [risk allele]=A; minor allele=G).

More particularly detection of the simultaneous presence of allele C of rs6872664 of PITX1, allele T of rs35678 of ATP2B2, allele C of rs2292813 of SLC25A12 and allele A of rs1861972 of EN2 is indicative of the presence of or predisposition to autism, or to an autism spectrum disorder.

The method of the invention, also referred to as “the test”, preferably includes genotyping of all four genes. The test can be used to strengthen the diagnosis by confirming a known risk profile. In such case a negative test result does not invalidate the diagnosis for autism.

Alternatively the test can be used to establish a detailed risk profile for the non-affected sibling. Possible outcomes are:
presence of a risk allele in one or more genes, heterozygous or homozygous implicating increased risk
absence of a risk allele in the un-affected sibling and/or the autistic sibling. In this case no risk profile can be established.

Interestingly, the inventors have further shown an additive effect of multiple risk alleles A particular diagnostic method of the invention thus comprises determining the number ofrisk alleles, wherein the more risk alleles are detected within the gene loci PITX1, ATP2B2, SLC25A12 and EN2 combined, the more increased is the risk of developing autism or a autism-spectrum disorder.

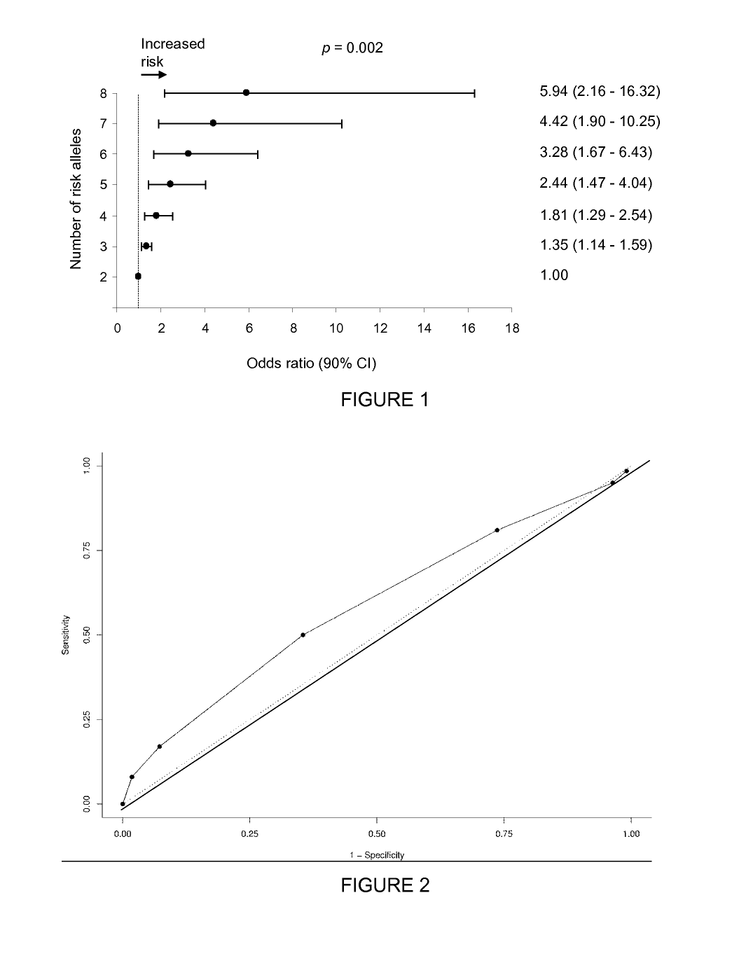

More particularly, tested subjects with 5 risk alleles or more can be classified as high-risk subjects (see FIG. 1).

The presence of an alteration in the gene locus may be detected by sequencing, selective hybridisation and/or selective amplification.

Sequencing can be carried out using techniques well known in the art, using automatic sequencers. The sequencing may be performed on the complete genes or, more preferably, on specific domains thereof, typically those known or suspected to carry deleterious mutations or other alterations.

Amplification is based on the formation of specific hybrids between complementary nucleic acid sequences that serve to initiate nucleic acid reproduction.

Amplification may be performed according to various techniques known in the art, such as by polymerase chain reaction (PCR), ligase chain reaction (LCR), strand displacement amplification (SDA) and nucleic acid sequence based amplification (NASBA). These techniques can be performed using commercially available reagents and protocols. Preferred techniques use allele-specific PCR or PCR-SSCP. Amplification usually requires the use of specific nucleic acid primers, to initiate the reaction.

Nucleic acid primers useful for amplifying sequences from the gene or locus are able to specifically hybridize with a portion of the gene locus that flank a target region of said locus, said target region being altered in certain subjects having autism, an autism spectrum disorder, or an autism-associated disorder

Hybridization detection methods are based on the formation of specific hybrids between complementary nucleic acid sequences that serve to detect nucleic acid sequence alteration(s). A particular detection technique involves the use of a nucleic acid probe specific for wild type or altered gene, followed by the detection of the presence of a hybrid. The probe may be in suspension or immobilized on a substrate or support (as in nucleic acid array or chips technologies). The probe is typically labelled to facilitate detection of hybrids.

In a most preferred embodiment, an alteration in the gene locus is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the alteration of the genes, a sample from a test subject is labelled and contacted with the microarray in hybridization conditions, leading to the formation of complexes between target nucleic acids that are complementary to probe sequences attached to the microarray surface. The presence of labelled hybridized complexes is then detected. Many variants of the microarray hybridization technology are available to the man skilled in the art (see e.g. the review by Kidgell&Winzeler, 2005 or the review by Hoheisel, 2006).

The examples illustrate the present invention without limiting its scope.

### EXAMPLES

### Example 1

### Autism Risk Prediction in Children

### Materials and Methods

### Study Design

The primary objective of this single center study with prospective genotyping was to evaluate the risk associated with 4 low-penetrance single nucleotide polymorphisms (SNPs) (rs6872664 [PITX1], rs35678 [ATP2B2], rs2292813 [SLC25A12], and rs1861972 [EN2]) in a multigene model in siblings of children diagnosed with autism, pervasive developmental disorder, or autism spectrum disorders (affected, broad phenotype). The alleles were as follows: rs6872664 (major allele [risk allele]=C; minor allele=T); rs35678 (major allele=C; minor allele [risk allele]=T; recessive coding for risk allele in risk score), rs2292813 (major allele [risk allele]=C; minor allele=T), and rs1861972 (major allele [risk allele]=A; minor allele=G).

Nuclear families with at least two offspring, at least one of which was affected by an autism spectrum disorder, were recruited from a variety of sources, including newspaper articles, parent organizations, and a network of community service providers. The sample included 295 families containing a total of 659 affected children (80.9% male); 276 families had at least 2 affected children; 19 families had one affected child. Approximately 50% of families were from the greater Seattle region. Children were categorized as affected based on Autism Diagnostic Interview Revised (ADI-R) (Lord et al, 1994) and Autism Diagnostic Observation Schedule (ADOS-G) (Lord et al, 2000) scores and on a clinical diagnosis by an experienced clinician. Diagnostic categories were autism (n=565; 81.6% male), pervasive developmental disorder (n=35; 77.1% male), and autism spectrum disorders (n=59; 76.3% male). Specific details regarding diagnostic criteria can be found in Schellenberg et al., 2006. Average chronological age at intellectual quotient assessment was 8.96±4.29 years (n=581); average composite intellectual quotient score was 76.94±26.32 (n=575) with 39.3% scoring below 70. Ethnicity, which was self- or parent-reported, was distributed as follows: Caucasian (73.4%), Asian (2.4%), Hispanic/Latino (2.4%), Black/African American (1.5%), American Indian/Alaska Native (1.5%), Native Hawaiian/other Pacific Islander (0.3%), more than one ethnicity (9.1%), and unknown/not reported (9.1%).

When the family contained other children who were not on the autism spectrum, one of these children was also assessed. This sibling was classified as unaffected based on the parent report, the Family History Interview (Bolton et al, 1998) and the Broader Phenotype of Autism Symptom Scale (Dawson et al, 2007). There were 162 (54.9%), 103 (34.92%), 20 (6.8%), 9 (3.1%), and 1 (0.3%) families with 2, 3, 4, 5 and 6 siblings per family, respectively.

Exclusionary criteria for affected children included a diagnosis of Rett syndrome and childhood disintegrative disorder as defined by the Diagnostic and Statistical Manual of Mental Disorders, Fourth Edition criteria for other pervasive developmental disorders, (American Psychatric Publishing, 1994) presence of a known genetic condition, history of serious head injury or neurological disease, or significant sensory or motor impairment (Schellenberg et al, 2006) Families were also excluded if they had previously participated in the Autism Genetic Resource Exchange (AGRE) program because previous association studies for these genes were mainly performed in AGRE samples.

This study was approved by institutional review boards. All subjects or their legally authorized representatives provided written informed consent.

### Genotyping

Samples were genotyped using TaqMan allele discrimination assays supplied by Applied Biosystems (Foster City, Calif., USA). Genotyping was performed on 384 well plates with 5 ng genomic DNA, 0.075 μl of 20× SNP TaqMan Assay mix, 1.5 μl of TaqMan Universal PCR Master Mix and 1.425 μl of dH₂O in each well. PCR was then carried out using a 9700 Gene Amp PCR System (Applied Biosystems) with a profile of 95° C. for 10 min and then 50 cycles at 92° C. for 15 sec and 60° C. for 90 sec. Plates were then subjected to end-point read in a 7900 Real-Time PCR System (Applied Biosystems). The results were first evaluated by cluster variations; the allele calls were then assigned automatically. Genotyping and data analysis were blinded to patient identification. Signal intensity plots and missing genotype frequencies were used for investigating genotyping quality. Poor clustering and missing fractions 5% per SNP lead to regenotyping. Genotyping success rate was 97.4%.

Parents were genotyped to check for Mendelian inconsistencies and to verify family relationships. All inconsistencies lead to regenotyping of the family. Families for which inconsistencies could not be resolved for at least one child, were excluded for that specific marker. Families for which there were unresolved inconsistencies for more than one SNP were also excluded. Deviations from and compatibility with Hardy-Weinberg Equilibrium were investigated for parents and control (unaffected) siblings (Ziegler et al, 2006).

### Statistics

### Primary Analysis

The primary analysis was performed on unaffected and affected siblings of the index case according to a written statistical analysis plan. The index case for the family was defined as the oldest affected child; index cases were used solely for inclusion in the study and were not included in the analysis. Adjustments for relatedness of siblings within families were performed using independence estimating equations.

Four primary analyses were performed according to the hierarchical test procedure described below. All primary analyses were conducted using SAS 9.1 (SAS Institute Inc, Cary, N.C., USA).

1) The effect of the total number of carried risk alleles was analyzed. The SNPs were used in an additive coding except for rs35678 from ATP2B2 which was coded recessive for the T allele. One-sided Wald-type p-values were estimated together with odds ratios (ORs) and 90% confidence intervals (CIs) for the increase in one risk allele. Analyses were done with adjustment for gender using the logistic link function and the binomial family.
2) The performance of the test overall was analyzed. This second primary analysis was only to be performed if the first analysis was significant at the one-sided 5% test level. A ROC curve was constructed. The AUC and 90% CIs were estimated using an independence estimating equations logistic regression model with the fully iterated jackknife estimator of variance. The AUC was tested against 0.5 (Dahmen et al, 2004).
3) Genotype effects were investigated. This third primary analysis was only to be performed if both the first and second primary analyses were significant. Genotype effects were investigated using one-sided Cochran-Armitage trend-tests for each SNP with adjustment for gender. Odds ratios and 90% CIs were calculated for the increase per risk allele. An adjustment for multiple testing of four SNPs was performed using the \check{S}idak-Holm step-down procedure.
4) For each possible combination of the number of carried risk alleles, the sensitivity, 1 minus specificity, and accuracy (“hit rate”) together with 95% CIs were tabulated.

### Secondary Analyses

Sensitivity analyses were conducted without formal statistical testing using two-sided tests and 95% CIs analogously to those described in the primary analyses. Specifically, using the standard logistic regression model, the inventors investigated, 1) the subgroup of Caucasian families, 2) all families according to the strict phenotype definition (autism but not autism spectrum disorder or pervasive developmental disorder), 3) all families without adjustment for gender, and 4) all families including only one sibling to the index case. These families included the first unaffected sibling if an unaffected sibling was available.

### Results

None of the SNPs showed deviation from Hardy-Weinberg Equilibrium at the nominal 5% test level for either parents or control siblings. Allele frequencies were similar to those reported in the HapMap database and previous publications (Benayed et al, 2005).

In the 590 founders, the risk allele frequency was 89.9% (95% CI: 87.5-91.6; n=1058) for the C allele of rs6872664 (PITX1), 40.5% (95% CI: 37.4-44.0; n=1060) for the T allele of rs35678 (ATP2B2); 90.2% (95% CI: 87.7-91.8; n=1062) for the C allele of rs2292813 (SLC25A12); and 73.0% (95% CI: 68.9-75.1; n=1024) for the A allele of rs1861972 (EN2).

When the additive effect of multiple risk alleles was investigated in the first primary analysis, the ORs increased significantly with the number of risk alleles (FIG. 1). The increase per risk allele was 1.35 (90% CI: 1.14-1.59; p=0.002). Children with 6 or more alleles, had an OR between 3.28 (90% CI: 1.67-6.43) and 5.94 (90% CI: 2.16-16.32). Children with 5 alleles or more, had an OR of 2.44 (90% CI: 1.47-4.04). The AUC for the corresponding ROC curve was 0.59 (90% CI: 0.54-0.63, p=0.001; FIG. 2).

Because none of the single markers were significant after adjustment for multiple testing (lowest nominal p=0.026; lowest adjusted p=0.100), the hierarchical testing procedure was stopped at the third primary analysis.

Results from the fourth primary analysis (sensitivity, 1 minus specificity, and accuracy) are presented in Table 1.

TABLE 1
Sensitivity and 1 minus specificity analysis according to
the specific risk score values: primary analysis.

| Risk | | 1-Specificity | |
|---|---|---|---|
| score | Sensitivity (95% CI) | (95% CI) | Accuracy (95% CI) |
| 2 | 0.99 (0.98-1.00) | 0.99 (0.97-1.00) | 0.70 (0.66-0.74) |
| 3 | 0.96 (0.94-0.98) | 0.95 (0.91-0.99) | 0.69 (0.65-0.73) |
| 4 | 0.83 (0.79-0.88) | 0.73 (0.65-0.81) | 0.67 (0.62-0.71) |
| 5 | 0.49 (0.43-0.56) | 0.37 (0.27-0.46) | 0.53 (0.49-0.58) |
| 6 | 0.16 (0.11-0.20) | 0.08 (0.02-0.13) | 0.38 (0.34-0.42) |
| 7 | 0.08 (0.05-0.11) | 0.03 (0.00-0.05) | 0.34 (0.30-0.38) |
| 8 | — | — | 0.30 (0.26-0.33) |

Fourth primary analysis: all siblings to the proband, broad definition of autism, which includes autism spectrum disorders and pervasive developmental disorders. No adjustment for gender. Point estimates and 95% confidence intervals (95% CI).

For 6 alleles, the specificity of the test was 92% and the sensitivity was 16%, whereas for 5 alleles, the specificity of the test was 63% and the sensitivity was 49%.

In the secondary analysis of the 4 subgroups, ORs increased with increasing numbers of risk alleles. Confidence intervals overlapped with those of the primary analysis.

### Discussion

Because studies in autism have shown that early intervention leads to improved treatment outcome (Fenske et al, 1985, Rogers et al, 1998), there is great interest in identifying and treating infants at risk for autism prior to onset of overt symptoms. Risk assessment based on testing for genetic polymorphisms associated with autism could allow interventions to begin during the infant period and thereby reduce or prevent the development of the full blown syndrome. The majority of single gene polymorphisms reported to date, however, carry low to moderate risk, with ORs rarely higher than Here, the inventors evaluated the clinical validity of a multigene risk assessment model in siblings of children with autism using a ROC curve with its associated AUC. The test combined four genes previously shown to be associated with autism.

The odds that a sibling of the index case would be affected by autism increased with the number of risk alleles the sibling carried. In the primary analysis, siblings carrying 8 alleles (homozygous for all 4 genes) had a significantly increased risk of being affected (OR of 5.94; 90% CI: 2.16-16.3) compared to those with two or fewer risk alleles. These data were supported by 4 planned subanalyses: 1) in Caucasians only, 2) in index cases affected by autism according to a strict definition, 3) with only 1 sibling to the proband included, and 4) with no adjustment for gender. As in the primary analysis, ORs increased with increasing numbers of risk alleles and confidence intervals overlapped with those of the primary analysis. Together these data suggest that the data in the primary analysis were stable and robust.

In order to evaluate the discriminatory power of this test, the inventors established a ROC curve for an increasing numbers of risk alleles. Here, they report an AUC of 0.59 (95% CI: 0.54-0.63), which shows that the test has significant discriminatory power (p=0.001) and that the results are significantly different from a random effect. In the context of other complex diseases, these data compare well to those from other multigene risk assessment models. Studies in type 2 diabetes mellitus and cardiovascular disease reported AUCs of 0.58 and 0.62, respectively (Weedon et al, 2006; Humphries et al, 2007).

Thus, the present data suggest that combining information from multiple risk polymorphisms provides a useful risk assessment tool for autism. The results obtained from the primary analysis confirm the clinical validity of this multigene test.

Although, in this study, the inventors analyzed the siblings of an index case, these data are likely to be applicable to most children presenting with autism symptoms. In fact, the family-based study design in which the controls were selected from within the families affected by autism, suggests that the control sample was enriched for the risk alleles. Consequently, in a case-control study in which the controls are unrelated to the probands, the ORs could be higher.

The results from this study should be viewed in light of the benefits of early intervention (Rogers et al, 1998).

A genetic test, which by its nature can be performed at any age, could provide physicians with a risk assessment tool that could complement existing clinical tools. Infants found to be at genetic risk for autism could then be more closely monitored so that interventions could begin as soon as early symptoms are observed. More specifically, such a use for this multigene genetic test would be in line with the American Academy of Pediatrics' recommendation to place children suspected of having an autism spectrum disorder in intervention programs as early as possible, even before a diagnosis is finalized (Johnson et al, 2007; Myers et al, 2007). This test could greatly shorten the time between suspicion of autism and early intervention and, ultimately, improve the outcomes of individuals suffering from this serious disorder.

### Example 2

The Tables below summarize the association between additional SNPs in the ATP2B2, EN2, PITX1 and SLC25A12 genes that may be used for diagnosing a risk for autism. P-values are given for two statistical models either additive or recessive. The frequency is provided for the risk allele, fam# denominates the number of informative families for each analysis.

### ATP2B2:

| | | Position | | | | | |
|---|---|---|---|---|---|---|---|
| SNP ID | Risk allele | (built 35) | Frequency | fam# · x | pvalAdditif | fam# · y | pvalRecessif |
| rs3774180 | G | 10371988 | 0.395 | 221 | 0.118742 | 142 | 0.003488 |
| rs775018 | G | 10375145 | 0.583 | 203 | 0.036013 | 179 | 0.005632 |
| rs28113 | A | 10375643 | 0.578 | 199 | 0.043877 | 178 | 0.007544 |
| rs2278556 | A | 10377103 | 0.387 | 219 | 0.018556 | 141 | 0.005703 |
| rs3774169 | T | 10391287 | 0.916 | 82 | 0.000602 | 84 | 0.000829 |

### EN2:

| | | Position | | | | | |
|---|---|---|---|---|---|---|---|
| SNP ID | Risk allele | (built 35) | Frequency | fam# · x | pvalAdditif | fam# · y | pvalRecessif |
| rs1861973 | A | 154753621 | 0.721 | 178 | 0.003671 | 182 | 0.029842 |

### PITX1:

| | Risk | Position | | | | | |
|---|---|---|---|---|---|---|---|
| rs# | allele | (built 35) | Frequency | fam# · x | pvalAdditif | fam# · y | pvalRecessif |
| rs6596188 | A | 134396002 | 0.882 | 103 | 0.017779 | 114 | 0.028901 |
| rs6596189 | C | 134396068 | 0.881 | 109 | 0.007245 | 120 | 0.014237 |
| rs6871427 | G | 134401926 | 0.872 | 94 | 0.00095 | 103 | 0.00338 |

### SLC25A12:

| | | Position | | | | | | |
|---|---|---|---|---|---|---|---|---|
| rs# | Allele | (built 35) | Frequency | Z · x | pvalAdditif | MAF · y | Z · y | pvalRecessif |
| rs13016580 | G | 172472035 | 0.702 | 1.886 | 0.059346 | 0.702 | 2.469 | 0.013547 |
| rs3770459 | G | 172474099 | 0.679 | 2.309 | 0.020937 | 0.679 | 2.728 | 0.006369 |
| rs1996424 | T | 172503872 | 0.678 | 1.814 | 0.06961 | 0.678 | 2.248 | 0.024589 |

### REFERENCES

Benayed R, Gharani N, Rossman I, et al. Support for the Homeobox Transcription Factor Gene ENGRAILED 2 as an Autism Spectrum Disorder Susceptibility Locus. Am J Hum Genet 2005; 77:851-68.
Bolton P F, Pickles A, Murphy M, Rutter M. Autism, affective and other psychiatric disorders: patterns of familial aggregation. Psychol Med 1998; 28:385-95.
Chakrabarti S, Fombonne E (2001) Pervasive developmental disorders in preschool children. Jama 285:3093-3099
Dahmen G, Ziegler A. Generalized Estimating Equations in Controlled Clinical Trials: Hypotheses Testing. Biometrical Journal 2004; 46:214-232.
Dawson G, Estes A, Munson J, Schellenberg G, Bernier R, Abbott R. Quantitative assessment of autism symptom-related traits in probands and parents: Broader Phenotype Autism Symptom Scale. J Autism Dev Disord 2007; 37:523-36.
Fenske E, Zalenski S, Krants P, McClannahand L. Age at intervention and treatment outcome for autistic children in a comprehensive intervention program. Special issue: Early intervention. Analysis and Intervention in Developmental Disabilities 1985; 5:49-58.
Filipek P A, Accardo P J, Baranek G T, Cook E H, Jr., Dawson G, Gordon B, Gravel J S, Johnson C P, Kallen R J, Levy S E, Minshew N J, Ozonoff S, Prizant B M, Rapin I, Rogers S J, Stone W L, Teplin S, Tuchman R F, Volkmar F R (1999) The screening and diagnosis of autistic spectrum disorders. J Autism Dev Disord 29:439-484
Flanders W D, Sun F, Yang Q (2001) New estimator of the genotype risk ratio for use in case-parental control studies. Am J Epidemiol 154:259-263
Fombonne E (2003) Epidemiological surveys of autism and other pervasive developmental disorders: an update. J Autism Dev Disord 33:365-382
Fombonne E, Cook E H (2003) MMR and autistic enterocolitis: consistent epidemiological failure to find an association. Mol Psychiatry 8:133-134
Hoheisel, Nature Reviews, Genetics, 2006, 7:200-210
Humphries S E, Cooper J A, Talmud P J, Miller G J. Candidate gene genotypes, along with conventional risk factor assessment, improve estimation of coronary heart disease risk in healthy UK men. Clin Chem 2007; 53:8-16.
Johnson C P, Myers S M. Identification and evaluation of children with autism spectrum disorders. Pediatrics 2007; 120:1183-215.
Kidgell&Winzeler, Chromosome Research, 2005:13:225-235
Lord C, Rutter M, Le Couteur A (1994) Autism Diagnostic Interview-Revised: a revised version of a diagnostic interview for caregivers of individuals with possible pervasive developmental disorders. J Autism Dev Disord 24:659-685
Lord C, Risi S, Lambrecht L, et al. The autism diagnostic observation schedule-generic: a standard measure of social and communication deficits associated with the spectrum of autism. J Autism Dev Disord 2000; 30:205-23.
Myers S M, Johnson CP. Management of children with autism spectrum disorders. Pediatrics 2007; 120:1162-82.
Philippi A, Tores F, Carayol J, et al. Association of autism with polymorphisms in the paired-like homeodomain transcription factor 1 (PITX1) on chromosome 5q31: a candidate gene analysis. BMC Med Genet 2007; 8:74.
Ramoz N, Reichert J G, Smith C J, et al. Linkage and association of the mitochondrial aspartate/glutamate carrier SLC25A12 gene with autism. Am J Psychiatry 2004; 161:662-9.
Rogers S J. Empirically supported comprehensive treatments for young children with autism. J Clin Child Psychol 1998; 27:168-79.
Schellenberg G D, Dawson G, Sung Y J, et al. Evidence for multiple loci from a genome scan of autism kindreds. Mol Psychiatry 2006; 11:1049-60.
Veenstra-VanderWeele J, Cook E H, Jr. (2004) Molecular genetics of autism spectrum disorder. Mol Psychiatry 9:819-832
Weedon M N, McCarthy M I, Hitman G, et al. Combining information from common type 2 diabetes risk polymorphisms improves disease prediction. PLoS Med 2006; 3:e374.
Ziegler A, König I. A statistical approach to genetic epidemiology: concepts and applications. Weinheim: Wiley-VCH, 2006.

1. A method of detecting the presence of or predisposition to autism, or to an autism spectrum disorder in a subject, the method comprising detecting the presence of an alteration in the gene loci of at least PITX1, ATP2B2, SLC25A12 and EN2 in a sample from said subject.

2. The method of claim 1, wherein the alteration is a single nucleotide polymorphism.

3. The method of claim 2, comprising detecting the presence of a single nucleotide polymorphism (SNP) at any of positions rs6872664, rs6596188, rs6596189 or rs6871427 of PITX1.

4. The method of claim 1, comprising detecting the presence of a single nucleotide polymorphism (SNP) at any of positions rs35678, rs3774180, rs775018, rs28113, rs2278556, or rs3774169 of ATP2B2 .

5. The method of claim 1, comprising detecting the presence of a single nucleotide polymorphism (SNP) at any of positions rs2292813, rs13016580, rs3770459, or rs1996424 of SLC25A12 .

6. The method of claim 1, comprising detecting the presence of a single nucleotide polymorphism (SNP) at any of positions of rs1861972 or rs1861973 of EN2 .

7. The method of claim 1, comprising detecting the simultaneous presence of a SNP at position rs6872664 of PITX1, rs35678 of ATP2B2, rs2292813 of SLC25A12 and rs1861972 of EN2 .

8. The method of claim 7, wherein detection of the simultaneous presence of allele C of rs6872664 of PITX1, allele T of rs35678 of ATP2B2, allele C of rs2292813 of SLC25A12 and allele A of rs1861972 of EN2 is indicative of the presence of or predisposition to autism, or to an autism spectrum disorder.

9. The method of claim 1, wherein the subject is a sibling of an individual with autism or an autism-spectrum disorder.

10. The method of claim 1, wherein the presence of an alteration in the gene locus is detected by sequencing, selective hybridisation and/or selective amplification.

11. The method of claim 1, wherein the presence of an alteration in the gene locus is determined by DNA chip analysis.

12. The method of claim 1, comprising determining the number of risk alleles, wherein the more risk alleles are detected within the gene loci PITX1, ATP2B2, SLC25A12 and EN2 combined, the more increased is the risk of developing autism or an autism-spectrum disorder.

13. The method of claim 2, comprising detecting the presence of a single nucleotide polymorphism (SNP) at any of positions rs35678, rs3774180, rs775018, rs28113, rs2278556, or rs3774169 of ATP2B.

14. The method of claim 3, comprising detecting the presence of a single nucleotide polymorphism (SNP) at any of positions rs35678, rs3774180, rs775018, rs28113, rs2278556, or rs3774169 of ATP2B.

15. The method of claim 2, comprising detecting the presence of a single nucleotide polymorphism (SNP) at any of positions rs2292813, rs13016580, rs3770459, or rs1996424 of SLC25A12.

16. The method of claim 3, comprising detecting the presence of a single nucleotide polymorphism (SNP) at any of positions rs2292813, rs13016580, rs3770459, or rs1996424 of SLC25A12.

17. The method of claim 4, comprising detecting the presence of a single nucleotide polymorphism (SNP) at any of positions rs2292813, rs13016580, rs3770459, or rs1996424 of SLC25A12.

18. The method of claim 13, comprising detecting the presence of a single nucleotide polymorphism (SNP) at any of positions rs2292813, rs13016580, rs3770459, or rs1996424 of SLC25A12.

19. The method of claim 14, comprising detecting the presence of a single nucleotide polymorphism (SNP) at any of positions rs2292813, rs13016580, rs3770459, or rs1996424 of SLC25A12.

20. The method of claim 2, comprising detecting the presence of a single nucleotide polymorphism (SNP) at any of positions of rs1861972 or rs1861973 of EN2.


SEQUENCE LISTING (16 sequences)

SEQ ID NO: 1 - DNA, 601 bp, Homo sapiens
  misc_feature (301)..(301): rs6872664=C/T
ccctccctccctccctctagggttcctgggaactgtccccactggaaagcgcccgccgggtgctggtcgtcagtaggcacctctagtctggtgtgccgcggagaagagcccaagaatcggagctggagccgcgggcctgcgttcctggctgggcagggcctgcacccttagctcggctcagattctgaccctgctgctgatgttcccagcaaatgtatgctttttgtttgtttgttttgcgaactcttagggggtctaaatctgagggggtctctgcttttctgaactaggatcagatctytccagcctaaagtccctccactttcttctcctcaggggtgtatgggagccccactgggcaggcacaacacagccgggttctcggccttgcttccaggcctccagtcccaaagcctagaaaaccccaccaactgcagcccagacagacagactctgtctaggtgacctcgggctttagtctgtctctctttgaagctacacaaaacacacgcagtcatagaaacattaatacataaagaaaaagagtttaaaaacaaaacacagaagcccagaaaagctcacagctctatgcctaaagaca

SEQ ID NO: 2 - DNA, 401 bp, Homo sapiens
  misc_feature (201)..(201): rs6596188=A/T
aagggccacaaattctcaaaatcagacaggaagaaacacagagcaaatgtaaattaagaagccagaaagctccgtacaaacacacccctaactaaatacagagctgtaagtgtataaactccaggttgacagaagcatttatttctctgggatttaaaaaaataatgtaccgttgtagatagatttttaactccttcccawtaaggcccttgtagagataatttttaaatcccagagaaacacattatagttagataatttaaattccccaaataacaccactgtaaatggattttgagaaaattccggagaaacgcgccattttggattgactttttaaaatctccggaagaataatgttgcatgtagatattttaaattccagacctacacttcgtacg

SEQ ID NO: 3 - DNA, 401 bp, Homo sapiens
  misc_feature (201)..(201): rs6596189=C/T
aagctccgtacaaacacacccctaactaaatacagagctgtaagtgtataaactccaggttgacagaagcatttatttctctgggatttaaaaaaataatgtaccgttgtagatagatttttaactccttcccaataaggcccttgtagagataatttttaaatcccagagaaacacattatagttagataatttaaattycccaaataacaccactgtaaatggattttgagaaaattccggagaaacgcgccattttggattgactttttaaaatctccggaagaataatgttgcatgtagatattttaaattccagacctacacttcgtacgttaattctttcagaatcgaaagatacagttaaaattaattcatcctcatagatttggttcaaaaaa

SEQ ID NO: 4 - DNA, 401 bp, Homo sapiens
  misc_feature (201)..(201): rs6871427=C/G
tggacgctgtctggttcctgagaatggctgagctgtggggaagaagactaggaaggaggtagcagctggtcgcacaactggggagcagaggggcaacttatggatgcaccatagcctcctcctcccagcggctgctttcatgggagaagagcaccctgagcctgtctggaggcctctgcatcctagagtccagacagcctsgtctccaagccccagcccacctcttttcctttccctggaagagatgcgggaggccgaaagcagccccgggaaagggcacggggacagcaagtctcctgtgctgcccctgctgcctgtagcttttgccctgactaccgtagacctgaaccctagacctgaatcctcccccagtggacagcaacctgggatttcctccctct

SEQ ID NO: 5 - DNA, 1001 bp, Homo sapiens
  misc_feature (501)..(501): rs35678=C/T
ggcggaccccctgaccaccacgccctgggggaccctgggcgacctggcctgcagcttctgtccagcctcacacggcccctgtgggtgggtgcccccagccctgagcgacagtcccctcactcacctctgccctcagagccacccttccccaccctctcagggaaatccccctggagaccacggtgggtgtgacccctaccacttccccgtgctgccctgagagggcacaatgggacctctctgaggaggccgtgctgttccttagcagccacaggagtgggtgtgcaagtggctggtgagagctggccgggacccctctgtagccaccctccctcagcaccagagctgggccctggggggcagcacaagcacactcacctccatcccctggcccgccccggccaggcctgggcccagcccccaagagcctcctgtacctgtgtctggatccgattcaggcctcggaaccacaggatctggccccgccgcagctcccgctcygcgtggtcgatctcctccacgtcctcgttgagctcctcctccgggatctcctccttctgtgtgagcctgcctgcctccttgaggaacttgagtctgctggtcgggatggtggcgatgacctgcaagggaccctgtctgtcaggacggtggggctgtccttcccagtggccttctctccctgacacccgctcctctacctccctcctcctacccgctcacccaggcatgtgatcaatctactccttcacccacctcacctgttcctcagcccagtctttgacaatgccttttttttttttttttttttttttgagatggagtttcactcttgttgcccaggctggagtgcaatttgctttttaattcattcattcagagtcagggtacatagtggacaatcttggcctctaggagttggacagatgtggctttgaataccccagacaaccctggtcctgtggcctcgggcaagtcatttaacctcaccgagctttggtttt

SEQ ID NO: 6 - DNA, 201 bp, Homo sapiens
  misc_feature (101)..(101): rs3774180=A/G
ctgttggctcacccctgggatgtctatataggagtcataaactgtctcacagcagcaacttactcaagtgacacagcagactcaagggacatgggttctarttctgcccctgccactccctcacagatggcctcagtttccccacttgaaaactgagagaatcagactgtccagcattcacagtgagggacacagcagggt

SEQ ID NO: 7 - DNA, 825 bp, Homo sapiens
  misc_feature (326)..(326): rs775018=A/G
aaaagtagactctcagctgtgttcctaggccagcttgctggggtaaactggctggtaaactgcatctgcactgggagaactataaatactgacagggacaccacacttcgggttctcctgagtgcaatgcccttgctattgggtgtgtcctttgtagggaccctggaatccatgtttgtggatttgttacaggagatcctggctcctgcagggcaagaggtttctaaggcagggcagcccctacacctgtcccttgggggaccagagggaagagctcatggtggctggctggacagctcgaggactgcccccatggcagagcaatrcctgctggcaacctgtgggttctgtgtccttctgggccgacccatgccacatgtggtctacactgtttctcatgggcaatcctcttatgaggatcaaggaaggaaatccccatgggcattatgtggaaactgagactcaaagaggtgaagtgacttactccaggtcaccctggtggctggcctcccagcctcatcccctttccctgagctcgctggccctggggcctacctctggccgcaccgggtcctcgatgcccaccacgcagatgcaggtgagttcgttgaggatgtcattctcattgtcccagtccggctccgggctgctggggaagtcgcggtaggccacgcagatagtgcggagcccatcgcaagccatgggctcaatcaccttctttaccatctcgtcccggtcgcggggccggaagacacgaggctctcccgccccattgaggattttgcagcacctaggggaggatggaagggagatggggagcccagg

SEQ ID NO: 8 - DNA, 801 bp, Homo sapiens
  misc_feature (401)..(401): rs28113=A/G
ggaaggaaatccccatgggcattatgtggaaactgagactcaaagaggtgaagtgacttactccaggtcaccctggtggctggcctcccagcctcatcccctttccctgagctcgctggccctggggcctacctctggccgcaccgggtcctcgatgcccaccacgcagatgcaggtgagttcgttgaggatgtcattctcattgtcccagtccggctccgggctgctggggaagtcgcggtaggccacgcagatagtgcggagcccatcgcaagccatgggctcaatcaccttctttaccatctcgtcccggtcgcggggccggaagacacgaggctctcccgccccattgaggattttgcagcacctaggggaggatggaagggagatggggagcccarggaatgctccttctgaaaggcttagagaccacctccccaccctcgtgatgcccagctagctgtggctggtcatccagtgcccatctagttcatcccccaggcagggtgaaatcaagcattcgtcctgcaatttttgcccaggccactcagcaagcggagccccactttgatgtattatatactcacaagattcatctgaaataacactcccacagctgaaatggtttacaaagcacataaaactcaggctagtagttcttgaccctgcctgcacattagaattgcttggaggagcctttaataaatacccacgtgcgagccttgccccagaggttccagttcaatttgtctggggtgaatccaggaatcaatgtttttttaaacactcctcagagtgatt

SEQ ID NO: 9 - DNA, 701 bp, Homo sapiens
  misc_feature (201)..(201): rs2278556=A/G
tggtctctgggctgggggctgagccctcagggagcccttggcccatcccctgcaccctcagggctctgcatcccaggtccagtcctgactccgggtgaaaggagtgaagcaagattagcttttaaaaaaattatgaattatttcaaacatgaaaaagagaatgatgtaacaaagttacgtgcctatcatccagctttgtarcatcttaacattatgccgtacttgcctcagatttttctattttaagaagtaaaacattacagataaagttgaatcccctccgcaaacctctccccatctgcataattcactccttgtcccagaaacaacttctgtcttaaatttagcattaatcattaccatgtgtgtatttaactgttaattccgagataattgtaattcacatgcaataagaaacaatacagagatgccgtatacccttcatccagtttcccccaatgtcatcaccatagtattaacacaagtaccacaactgagatggagatgctgacgcaaggaagccgaacttgtggagagttcaccggctttacatgagcttgtgtgtgggtgtgcgtgtgtattttctatgcaatggcaccacatgtgagttaatgtggccaccaccactgtcaagattcaggacagttcatcacaagggtccttgtactaccctttttttaaagctatggtcacctctctct

SEQ ID NO: 10 - DNA, 201 bp, Homo sapiens
  misc_feature (101)..(101): rs3774169=C/T
tgcagtgcccagtcactgcgagtctgaccctactgggtcacccccaggttctcacacccgggtctcattttcttccctttgccccatgttttctccacctygaagggccgggcgtagggcttctgtttcccagtgccccatgccagaatgtccctgcaagggtggaggcgtggccatgtttataaacgaggcattcactct

SEQ ID NO: 11 - DNA, 801 bp, Homo sapiens
  misc_feature (401)..(401): rs2292813=C/T
gtcttttttaaaaaaatttgctgcttccctaaaatcccccaaatagctgcttaatattatgtacgatgtcactctgtcaagttttacttgcttaacacaaatgcttcttaagctcactgttttaaatgcattggtcttaaaggttctgtcttatcagttttctaagagttgcaacaatgcttttgtagacagaacaaacatttgttttggacttagtgatttctttgtaaaggagttgagcagctgacttacaggcctccaaaatcaatgtaaaaccaccgctggagaagttcataagtgaccaaggtaacaccaaactggggagaggatcgaaacactcgagctgaaaaagagaagcaggggcaggggagacttgaaaccaggacaaatgtggtaaagayagccactgagtcacaggggaggacgctagagccagccagccattctgtatggctccagcccctgcctacctgcagtccctttccaaaatgctgagggcccttcttcccggagaatcttcctgaaacagtcgatgacaccactgtatgtcgtctggccagcgcgggcagccacctgcagtcttgtcttgatgacatcagcaggggtcaccagagatgcagctgggacacctttcaggagaaaaccacatttaatttatctagagtaccttataaaagataggaatactgctaacatctactatagagcctgcaacttcaaccacttgagcggaactcaaaactctttacatagatgtctggccattaatgggaaaagggcatagaagcctattagctgttg

SEQ ID NO: 12 - DNA, 620 bp, Homo sapiens
  misc_feature (420)..(420): rs13016580=A/G
ccaacactccgaaaagacaaaggaaggaaggggtgcatgtttgctctctatacaaccagtaatgaaccattacagagctcagtgtggctctgaccagctgtgctggccaacatggtgaaaccctgtctttactaaaaatacaaaaattggccaggcatggtggtacacgcctgtagtcccaactacttgggaggctgacacaggagaatcgcttgaacccgggaggcagaggttgcagtgagcagagattgtaccactgcactccagcctgggtgacagagcaagattctgtctcaccaaaaaaaaaaaagtgggagactggtaagtaccagaaaggggctgctacaaacagctcactgccaagactgagcaggtaactttactggcttttaaacacaaaactgactaaaattaccattrctagaggagaaaggacttgctgggccccaaataagggcggtaagttccctggggccgtttcttaatgatcaaagtcatccctgtcttcacgagcaagaacaacgtggtccatcgcaacactctcaggcccaagggctcctaccacacaacaagcacagcacactccgggagctgctcaggttgccctgatgtttagccag

SEQ ID NO: 13 - DNA, 601 bp, Homo sapiens
  misc_feature (301)..(301): rs3770459=G/T
tcagacggtcaaggctacagcaagctgtgatcctgccactatgctccagcctgggcaacatagtgagaccctgtctcagaaaaaaaaaaaaattaaaaagaataaaacagagtcattaaaaaaacaagcgccaaggtggagtgtttgcctagctaggtagtggaatggtgtgggcttctgacaaagctgagggaggtgttctttgagacctcagtcattgggacaagctgcccctgggtaatgattttctactcctgtggaaacctgccgctaaaatattttgtctgctatttaggacatkgggaatgaggagagaagatttgggtttcttttccccctgctctagagtagttacttttccttcaccactgcctatagctttttacctggcactgcggggactctcagctgatgtaaaaacttaagagggtagatcttgtgtgtttttaccatagtaagagattaattaattttaaaaactctgaatctatataagaaatgccagaaagttcttagttttcagaatactatcattacccagttattaactctaggactgcaggattcaccatgctttcacaagctttgagatatgcagatt

SEQ ID NO: 14 - DNA, 601 bp, Homo sapiens
  misc_feature (301)..(301): rs1996424=A/T
cagttttaatgtgtgttttcttgtactaatgatattaaacttttctattatttttccttaccatttggatatcctcttttgtgaggtttctgttcaggtatttagccaatttttctattgcattgcctgccttttgcttataaatttgcacaagtttattatactatatattctggatacagatcctctattatttaaattattgcaaatatctctgcctattgtgtaacttgtcttttcacactcttagtggtatattttgataaaagttcttaaatctaatgaagtttaacttattaawctttttctttatgactggtgttctgtactattcacaaaatctctgcctatctccagatcataaatagaatatcttgtgttatcctctttaatctttcacatttaggaacaggaaaagacttttgtgtatggtatggggtagtgattttttttccttgttgaatatccatcattaattaaaaatttttaaaaacaggcctggtaaaatttcagccagcatcaaaaaattaaaatataaaacatttcaggcacttaaaaaatacaaagatatattaatactttttcctcacttcccatcccc

SEQ ID NO: 15 - DNA, 601 bp, Homo sapiens
  misc_feature (301)..(301): rs1861972=A/G
agggagtgcttggcattcaccctgggcctccagttcgtcccccacctcttgctgggcacagccccagcaccctagttgactctcctgacctgggcagggtgcagtcccagggcctccaaggagatccacattcctcttctcctcagtgtgcccggcagctctccggccctgaagggtggggggcccccagccttctccagccacagggacctgtgatgaagctggggccagatgctccctaaagccgattcatacaccgcacaaattgaaacccagaggcgaggtcaccactccctgccartggccttgcccccttcttcccccacagggaacgccagggggttgagcctcttatcaccaaaaagaaactgatgacacttccctccttctgctctcctccctctgccctttccccatggatagcaggtcctagaagccttacagcgaccctgcccaaaacctggggcaggtccacagggagaaggccaggtcaggttcataagtctgaatcccagttgggaggcacagtggggagggtcagaagtggacctggacaaggtcagctgggctaccctgctgcccacagtgaagcagtcccatg

SEQ ID NO: 16 - DNA, 601 bp, Homo sapiens
  misc_feature (301)..(301): rs1861973=C/T
cggcagctctccggccctgaagggtggggggcccccagccttctccagccacagggacctgtgatgaagctggggccagatgctccctaaagccgattcatacaccgcacaaattgaaacccagaggcgaggtcaccactccctgccaatggccttgcccccttcttcccccacagggaacgccagggggttgagcctcttatcaccaaaaagaaactgatgacacttccctccttctgctctcctccctctgccctttccccatggatagcaggtcctagaagccttacagcgaccctgyccaaaacctggggcaggtccacagggagaaggccaggtcaggttcataagtctgaatcccagttgggaggcacagtggggagggtcagaagtggacctggacaaggtcagctgggctaccctgctgcccacagtgaagcagtcccatgtctggggaaagggtggtgcagtcaacacccttgtagagccaggtcccctctcctggacaggaaacctgggagatttccagtgggtgaaggactcacccactgtgagtagctcagtgcccctccccaccaaggagggaagtacatgcactgac